(19) United States
(12) Patent Application Publication
(10) Pub. No.: US 2005/0209571 A1
(43) Pub. Date: Sep. 22, 2005

(54) Syringe with split/adjustable thumb ring

(76) Inventor: William J. McKay, Gurnee, IL (US)

Correspondence Address: WELSH & KATZ, LTD, 120 S RIVERSIDE PLAZA, 22ND FLOOR, CHICAGO, IL, 60606, US

(21) Appl. No.: 11/080,762

(22) Filed: Mar. 15, 2005

Related U.S. Application Data
(60) Provisional application No. 60/553,434, filed on Mar. 16, 2004.

(51) Int. Cl. A61M 5/315

(52) U.S. Cl. 604/227

(57) ABSTRACT

A small dental syringe has a spring biased plunger and a split, adjustable thumb receiving member. The dimensions of the syringe result in a shorter, over-all, distance a user must spread his/her thumb from the fingers gripping the syringe for aspirating.

### CROSS REFERENCE TO RELATED APPLICATION

This application is based on and claims priority of U.S. Provisional Application Ser. No. 60/553,434 filed Mar. 16, 2004 and entitled “Syringe with Split/Adjustable Thumb Ring”.

### FIELD OF THE INVENTION

The invention pertains to dental syringes.

### BACKGROUND OF THE INVENTION

Known dental syringes usually incorporate a hollow body which is intended to receive a fluid containing vial, for example, anesthetic. The vials are generally in the form of a cylinder with seals at each end.

One end of the housing carries a harpoon affixed to an elongated plunger which terminates at a proximal end in a circular, continuous thumb ring. The other end of the housing carries threaded fixture to receive a hollow needle. The needle can be screwed, for example, onto the distal end of the housing. The needle has a distal, sterile end which is intended to penetrate patient tissue for the purpose of injecting the contents of the vial. A proximal end of the needle incorporates a vial penetrator which is a short needle.

As those of skill in the art will understand, the vial is inserted into the hollow handle. The harpoon is driven into the proximal end of the vial and interacts with a slidable, usually rubber seal. If desired, the harpoon can be driven into the vial prior to coupling the needle to the distal end. Once the needle has been coupled to the distal end the vial penetrator penetrates the distal end seal of the vial and provides a flow path to the distal end output of the needle. When the dentist pushes on the thumb ring, the rubber seal then forces the medicant from the vial through the needle into the tissue of the patient.

Known dental syringes while generally effective for their intended purpose have exhibited certain deficiencies in use. Generally, they have been sized and dimensioned for individuals, for example, males with relatively large hands. Unfortunately, many people who are practitioners in the field of dentistry in various capacities have smaller hands.

The known syringes can cause discomfort to the hands of the dental practitioner where it is necessary to stretch the portion of the hand between the thumb and the first and second fingers for the purpose of managing both the thumb ring and the proximal end of the syringe. This is not only undesirable from the point of view of the general health of the hands of the practitioner, it also is undesirable in that it makes it more difficult for the practitioner, dentist or surgeon, to precisely manipulate the thumb ring. For example, prior to fully injecting the dosage of fluid in the vial into the patient's tissue, it is standard practice to aspirate by pulling back on the thumb ring so as to determine the position of the point of the needle, the distal end thereof, relative to the patient's adjacent blood vessel(s). It is recognized that for a variety of reasons if blood has been pulled back into the syringe, then the practitioner will want to find another location as the injection point. This avoids injecting anesthetic into the blood stream via the adjacent blood vessel.

In FIG. 1 a user is holding a prior art syringe with two fingers and with the user's thumb extending through the thumb ring. As the diagram of FIG. 1 illustrates, an individual with relatively small hands has a thumb which will not fill the hole in the thumb ring as would be the case of a person having larger hands. In addition, aspiration requires that the user pull back his/her thumb (and the ring) relative to the fingers gripping the syringe. As a result, a person having smaller hands must spread their thumb and fingers apart more so than someone with a larger hand when attempting to retract the thumb ring and the plunger, for aspirating prior to injecting the contents of the vial. This in turn results in undesirable stresses and strains on the hands of the user.

Thus, it would be desirable to be able to cost effectively address the smaller size hands of many practitioners, who find the known syringes to be uncomfortable or overly large. Preferably such improved syringes could address outstanding ergonomic needs of such practitioners to provide a comfortable to use syringe that provides for precise location of the treatment applying end of the instrument, as well as being of a comfortable configuration for pulling back on the plunger for purposes of observing if any blood has been pulled into the syringe and also for being of a configuration that makes for a smooth, controllable injection of the contents of the vial into the patient's tissue, thereby also improving patient comfort. Any such syringe should also be able to use standard 1.8 ml vials.

### BRIEF DESCRIPTION OF THE DRAWINGS

FIG. 1 is an overall view of a user holding a prior art syringe;

FIGS. 2A, 2B and 2C taken together are three different views of a syringe in accordance with the present invention;

FIG. 3 is an exploded view of the instrument of FIG. 2A, 2B or 2C;

FIG. 4 is a view of an alternate form of a syringe in accordance with the invention;

FIG. 5 is an exploded view of the syringe of FIG. 4; and

FIG. 6 is a view of a user holding the syringe of FIG. 4 and illustrating the benefits of the present invention.

### DETAILED DESCRIPTION OF THE EMBODIMENTS

While embodiments of this invention can take many different forms, specific embodiments thereof are shown in the drawings and will be described herein in detail with the understanding that the present disclosure is to be considered as an exemplification of the principles of the invention and is not intended to limit the invention to the specific embodiment illustrated.

A syringe that embodies the present invention accommodates a growing number of clinicians with small hands. Historically, aspirating syringes were designed with the idea that “one size fits all”. Generally, this was true at a time when most clinicians were male with male size hands. They performed all of the procedures requiring aspirating syringes.

With more women becoming dentists, and Hygienists being licensed to administer anesthetics, there has been a continuing need for a small syringe, able to use a standard 1.8 ml cartridge.

When aspirating with a syringe, the clinician is required to use his/her thumb to pull back on the plunger. As the aspirating procedure continues, the thumb is required to pull back even further. This can become a strain on the thumb and hand in general. If one has small hands this can be a very difficult procedures.

In another aspect of the invention, the present syringes can be made from stainless steel, rather than plated brass. The stainless steel syringe provides an easy to care for product that resists discoloration and/or tarnishing over multiple sterilization life cycles.

A syringe in accordance with the invention preferably has a somewhat shorter body than do known syringes. Further, a proximal concave portion of the body, usually held between first and second fingers by a practitioner, has a smaller, central diameter than is the case with prior art syringes. Instead of a continuous circular thumb ring as usually found in known syringes, a syringe in accordance with the invention includes a split adjustable handle.

In a disclosed embodiment of the invention, the split adjustable handle has a linear region which is attached to the plunger of the syringe. Extended from the linear region, at each end thereof, are curved handle regions which curve away from the linear region and toward one another, but terminate with a space therebetween. The space between the terminations of the end regions makes it possible for the practitioner to adjust the size and shape of the plunger handle for maximum comfort.

One of the advantages of a syringe which embodies the present invention is the fact that it incorporates a shorter barrel into which the vial is to be inserted. The shorter barrel, in turn, produces a shorter plunger which translates into less displacement needed between the handle for the thumb and the proximal end of the syringe for aspiration to check for the presence of blood. Additionally, the shorter plunger translates into less displacement between the thumb and the two fingers which typically hold the syringe.

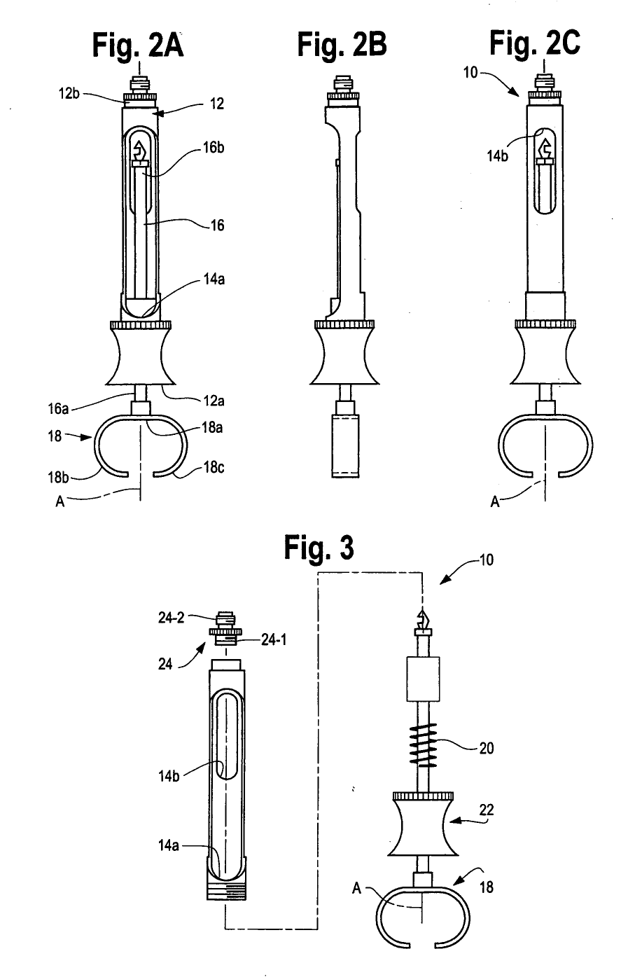

FIGS. 2A, B and C illustrate three different views of a syringe 10 in accordance with the present invention. The syringe 10 incorporates a barrel 12 which has a proximal end 12a and a distal end 12b. The barrel 12 is hollow and carries first and second slots 14a, 14b. The slot 14a has a length on the order of 62 mm. This length substantially corresponds to the length of known 1.8 ml medication vials.

The syringe 10 also incorporates a retractable plunger 16 which has a proximal end 16a, and distal end 16b.

An adjustable handle generally indicated at 18 is carried at the proximal end 16a of the plunger 16. The handle 18 includes a linear region 18a attached to the proximal end of the plunger 16a. Extending from the linear region 16a are curved end sections 18b, c.

The end sections 18b, c curve away from the proximal end 16a of the plunger and toward each other relative to a central axis A. The free ends of the sections 18b, c are spaced apart from one another thereby providing a handle which can be bent and adjusted to accommodate the size and shape of the user's thumb.

FIG. 3 is an exploded view of the instrument 10 illustrating the relative relationships of various components therein. The instrument 10 also incorporates a spring 20 which exerts a force which opposes attempts by the user to retract the handle 18 for aspiration.

A concave cylindrical member 22 is threaded onto the proximal end of the body 12. The member 22 has a smaller diameter than similar structures in prior art syringes.

A threaded fixture 24 is coupled to a distal end 12b of the body 12. The fixture 24 can, for example, be threaded into the distal end 12b using a first set of threads 24-1. A second exterior set of threads 24-2 is available to threadably and removably receive a needle. The fixture 24 has a boring therethrough which receives a penetrator end of the needle for purposes of piercing the distal end of the vial inserted into the body 12.

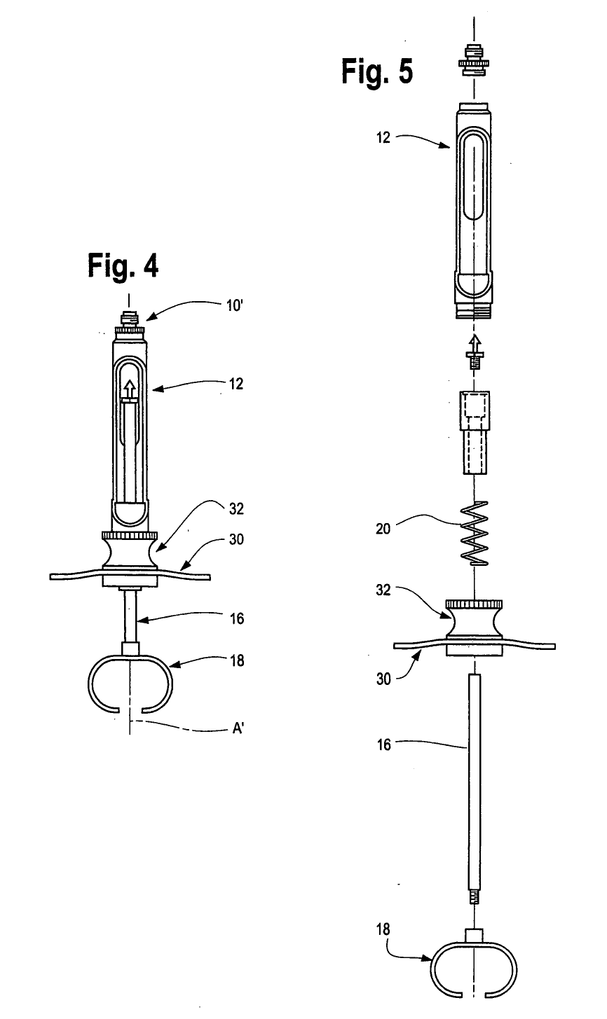

FIG. 4 illustrates an alternate syringe 10′ in accordance with the invention. The syringe 10′ incorporates a plurality of components which correspond to components of the instrument 10. Common components have been identified with the same identification numerals as used in FIGS. 2A, B, C and FIG. 3. The instrument 10′ carries at a proximal end thereof an elongated handle 30 adjacent to concave proximal end 32 which is attached to the body 12. The elongated gripping handle 30, in combination with concave proximal end member 32 provides an alternate to the concave member 22 of instrument 10.

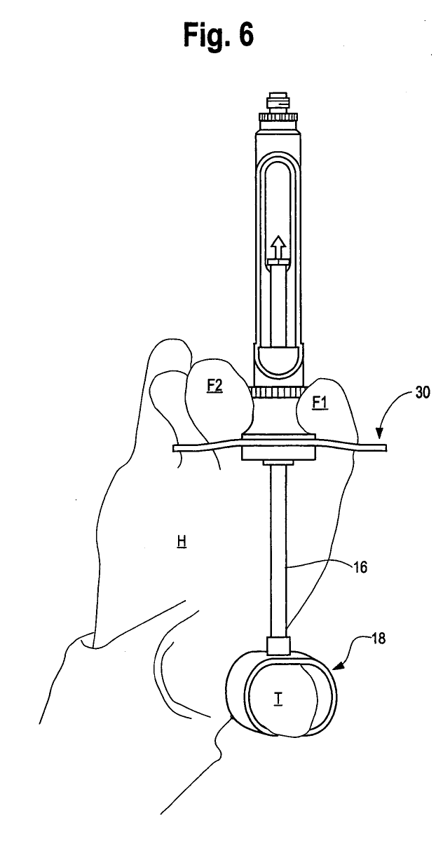

FIG. 6 illustrates a hand H of a user holding the instrument 10′ and displaying the advantages of a syringe, such as the syringe 10′ which embodies the present invention. As illustrated in FIG. 6, the fingers of the user F1, F2 grip the concave region 32 adjacent to the elongated handle portion 30.

To retract the plunger after an initial stick of the patient's tissues, the user's thumb T in combination with the fingers F1, F2, retract the plunger 16 using the handle 18. The shorter plunger in turn results in less displacement required between the fingers F1, F2 and the user's thumb T. This in turn improves user's comfort when using instruments 10 or 10′.

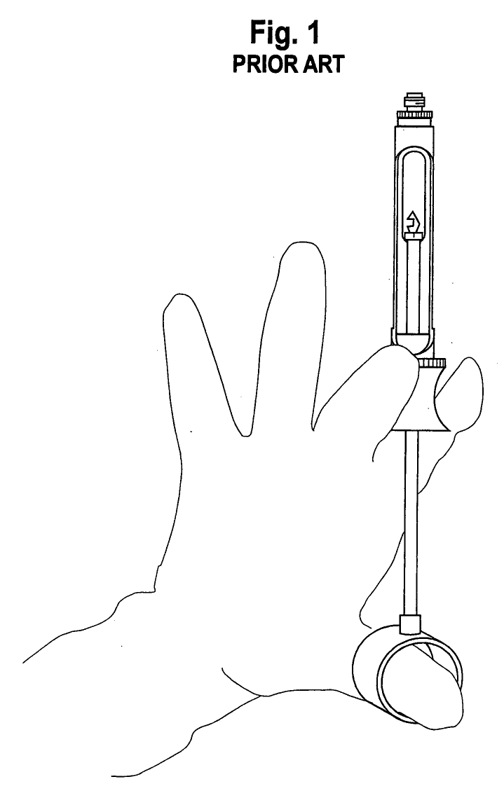

Table 1 illustrates differences in dimensions between instruments which embody the present invention, such as instruments 10, 10′ and the prior art instrument of FIG. 1.

As illustrated in Table 1, the shorter barrel 12 of the instruments 10, 10′ results in a shorter plunger and less thumb movement required for pull back and injection of the medication or anesthetic in the vial carried in the barrel 12.

TABLE 1
Present Syringes vs. Prior Art Syringes

| Features | Present Syringes | Prior Art Syringes |
|---|---|---|
| Overall Body length | 119 mm | 135 mm |
| Drum dimensions (width) | 15 mm | 20 mm |
| Drum dimensions (diameter) | 18.5 mm | 23.6 mm |
| Cartridge Size | 1.8 ml | 1.8 ml |
| Plunger Length (in open condition) | 58 mm | 60 mm |
| Length from inside top of ring | 76 mm | 87 mm |
| to top of drum | | |
| Window opening dimension | 62 mm | 67 mm |
| Ring Opening | 16 mm | 25 mm |
| Materials | Stainless | Plated Brass |

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

1. An apparatus comprising;
an elongated hollow body with at least one elongated slot therein;
an axially movable member with a proximal end and a distal end, the member is slidably carried by the housing with the distal end axially movable in the hollow body, the member carries an adjustable handle at the proximal end, the handle having a partially curved cross-section and extends from the body.

2. An apparatus as in claim 1 where the handle is attached by a central section to the proximal end, the handle includes first and second end sections which extend toward but are displaced from one another.

3. An apparatus as in claim 2 where the sections are deflectable to adjust the size of handle.

4. An apparatus as in claim 3 where in response to applied lateral forces, the sections move toward one another to alter a size parameter of the handle.

5. An apparatus as in claim 4 where the handle comprises an adjustable thumb ring.

6. An apparatus as in claim 1 where the body has first and second ends, one end is adjacent to the distal end, the other end is adjacent to the proximal end, the one end carries a set of threads.

7. An apparatus as in claim 6 which includes a hollow fluid infusion needle removably coupled to the threads.

8. An apparatus as in claim 7 which includes a fluid carrying cylinder slidably received in the hollow body when the distal end has been moved to the other end.

9. An apparatus as in claim 1 where the body includes a convex end adjacent to the proximal end.

10. An apparatus as in claim 1 where a cross-section of the adjustable handle has a linear section with curved deformable ends which extend toward one another and terminate displaced from one another.
